(19) United States
(12) Patent Application Publication
(10) Pub. No.: US 2025/0052024 A1
(43) Pub. Date: Feb. 13, 2025

(54) A SYSTEM AND METHOD TO MEASURE A DEFORMATION OF A GEOMATERIAL PORTION DUE TO COMPACTION OF THE GEOMATERIAL PORTION

(71) Applicant: Monash University, Clayton (AU)

(72) Inventors: Amir Tophel, Victoria (AU); Jayantha K. Kodikara, Victoria (AU); Jeffrey P. Walker, Victoria (AU)

(73) Assignee: Monash University, Clayton (AU)

(21) Appl. No.: 18/719,946

(22) PCT Filed: Dec. 17, 2021
(86) PCT No.: PCT/AU2021/051505

§ 371 Date: Jun. 14, 2024

(51) Int. Cl. E02D 1/02; E01C 23/01; G01B 21/32

(52) U.S. Cl. CPC E02D 1/022; E01C 23/01; G01B 21/32

(57) ABSTRACT

A system including:
a distance sensor system located/oriented/configured to measure a deformation of a geomaterial portion due to compaction of the geomaterial portion; and
an electronic processing system configured to:
receive signals from the distance sensor system representing the measured deformation, and
automatically generate an estimate/measure of a geomaterial layer property of the geomaterial portion based on:
the measured deformation; and
a pre-defined relationship/model that associates deformation values with geomaterial layer property values (e.g., density values, stiffness values, modulus values, energy values, or layer thicknesses during compaction).

### BACKGROUND

The majority of construction works require estimating the competence of the ground condition of a construction area (including geomaterial layers, e.g., soil) on which a structure is to be built, e.g., a building or a road. For example, if the soil is loosely compacted, a structure constructed over it will not function satisfactorily, and, in case of road construction, the service life of the road is reduced leading to premature failure. The quality assurance (QA) of engineered soil compaction is normally achieved by constructing the geomaterial layers to achieve a designated density and/or other geomaterial layer property values (e.g., deformation values, stiffness values, modulus values, energy values, or layer thicknesses during compaction). In addition, it is important to reduce/mitigate geomaterial variability within the geomaterial layers to reduce serviceability failures of the structure due to excessive differential deformations.

Pre-existing QA measurements may have issues/limitations. For example, invasive methods require sampling or disturbing the area by hammering or inserting a measurement system. The sampling area may need to be filled with soil and recompacted manually. Invasive methods include sand replacement, rubber balloon density, and borehole shear testing, which are lag indicators of the density because it can take around 2-7 days for the result to be available, and the depth of excavation is limited to about 300 mm. Pre-existing measurements of ground density have been made using a nuclear density gauge (NDG), which however emits harmful radiation and is slow to use because of pointwise measurements, e.g., making many measurements over a large area as multiple layers are applied and tested.

It is desired to address or ameliorate one or more disadvantages or limitations associated with the prior art, or to at least provide a useful alternative.

### SUMMARY

Described herein is a system 100 (for estimating/measuring properties, including density, stiffness, modulus, energy, or layer thickness, of a geomaterial layer 102 due to compaction by a compactor 104) may include:
a. a distance sensor system 302 located/oriented/configured to (continuously/continually/repeatedly) measure a (surface) deformation Δ_{N} (or height change) of a geomaterial portion (of a construction area or a mining area) due to compaction of the geomaterial portion (including measuring during compaction of the construction area or the mining area); and
b. an electronic processing system 106 configured to (continuously/continually/repeatedly):
   i. receive signals from the distance sensor system 302 representing the measured deformation (“deformation signals”), and
   ii. automatically generate a (numerical) estimate/measure of a geomaterial layer property (e.g., density values, stiffness values, modulus values, energy values, or layer thicknesses during compaction) of the geomaterial portion based on:
      1. the measured deformation (including continuously/continually/repeatedly during the compaction of the construction area or the mining area); and
      2. a pre-defined relationship/model that associates (surface) deformation values with geomaterial layer property values (e.g., density values, stiffness values, modulus values, energy values, or layer thicknesses during compaction) e.g., including a correlation relationship/model and/or a trained machine-learning model.

The distance sensor system 302 is configured/mounted/arranged to measure the deformation without touching (or being in physical contact with) the geomaterial portion.

The system 100 may include a motion/orientation sensor system (located on a movable platform with the distance sensor system, including on the rigid body 114 of the movable platform) and mounted/oriented/configured to (continuously/continually/repeatedly) measure motions/orientations of the movable platform synchronously with the measurements of the distance sensor system 302 (when the movable platform is moving).

The electronic processing system 106 may be configured to (continuously/continually/repeatedly):
a. receive signals from the motion/orientation sensor system representing the measured motions/orientations (“motion/orientation signals”), wherein the motion/orientation signals are synchronous with the deformation signals, and
b. automatically generate the estimate/measure of the geomaterial layer property of the geomaterial portion based on:
   i. the measured deformation;
   ii. a (numerical) correction based on the measured motion/orientations; and
   iii. a pre-defined relationship/model that associates (surface) deformation values with geomaterial layer property values (e.g., density values, stiffness values, modulus values, energy values, or layer thicknesses during compaction); and

The system 100 may include a geolocation unit 118 (or “positioning system”) located/mounted/oriented/configured to (continuously/continually/repeatedly) measure/determine a geolocation (a location representing latitude and longitude in coordinates of the construction area or the mining area) of the geomaterial portion synchronously with the measurements of the distance sensor system.

The electronic processing system 106 may be configured to (continuously/continually/repeatedly):
a. measure/determine the geolocation of the geomaterial portion so that the height of the geomaterial portion at same location can be measured after respective first and second compactions to generate a first, a second (and subsequent) measured heights (thus the height at a location is measured after every pass, and height measured at each pass is subtracted from the previous pass to calculate the deformation from each pass); and
b. receive signals from the distance sensor system representing the first and second measured heights of the geomaterial portion after respective first and second compactions, and
c. automatically generate a (numerical) estimate/measure of the geomaterial layer property (e.g., deformation, density, stiffness, modulus, energy during compaction, or layer thickness) of the geomaterial portion based on:
   i. the first and second measured heights (including determining/estimating the deformation from a numerical difference between the first and second measured heights); and
   ii. the pre-defined relationship/model representing a relationship between (surface) deformation values and geomaterial layer properties.

The electronic processing system 106 may be configured to (continuously/continually/repeatedly): generate a coded/color-coded map of geomaterial layer property values of a plurality of geomaterial portions (and thus up to the entire construction area or the mining area) based on the geolocations and their respective geomaterial layer property values. The determined/calculated/estimated geomaterial layer property values may be displayed in real time, e.g., to an operator of the compactor, e.g., to determine when compaction has reached a specified level for the construction area or the mining area. To this end, one or more machine-readable data-processing modules 410 may include a display module configured to generate the coded/color-coded map 500 of the geomaterial layer property values of the construction area or the mining area. The electronic processing system 106 may include an audio/visual component (“AV component”, e.g., a speaker and/or visible display) that is controlled by the display module to display the coded/color-coded map 500 of the area based on the measured/estimated geomaterial layer property values for the area, e.g., including respective measured/estimated geomaterial layer property values for portions of the area. The map 500 may display whether the respective measured/estimated geomaterial layer property values are above or below one of the threshold geomaterial layer property values, thus visually indicating whether the measured/estimated values for the portions are sufficient for the pre-defined QA specification, and thus whether further compaction is required.

The distance sensor system 302 may include: at least one first distance sensor 108 (also referred to as a “first sensor arrangement”) located/oriented/configured to measure at least one first height of the geomaterial portion before compaction (of the geomaterial portion); and at least one second distance sensor 110 (also referred to as a “second sensor arrangement”) located/oriented/configured to measure at least one second height of the geomaterial portion after the compaction (of the geomaterial portion). The distance sensor system 302 or the electronic processing system 106 may be configured to determine the measured deformation from a difference between the measured first height and the measured second height.

The first distance sensor 108 may be configured for attachment to a first side (in front) of a compactor element 112 (e.g., the roller), with the second distance sensor 110 configured for attachment to a second side of (behind) the compactor element 112, such that the first distance sensor 108 can measure the first height before the compaction (wherein the compactor element 112 operates to compact the geomaterial portion by moving across the geomaterial portion) and the second distance sensor 110 can measure the second height after the compaction.

The motion/orientation sensor system may include a motion/vibration sensor system and/or an orientation/angle sensor system.

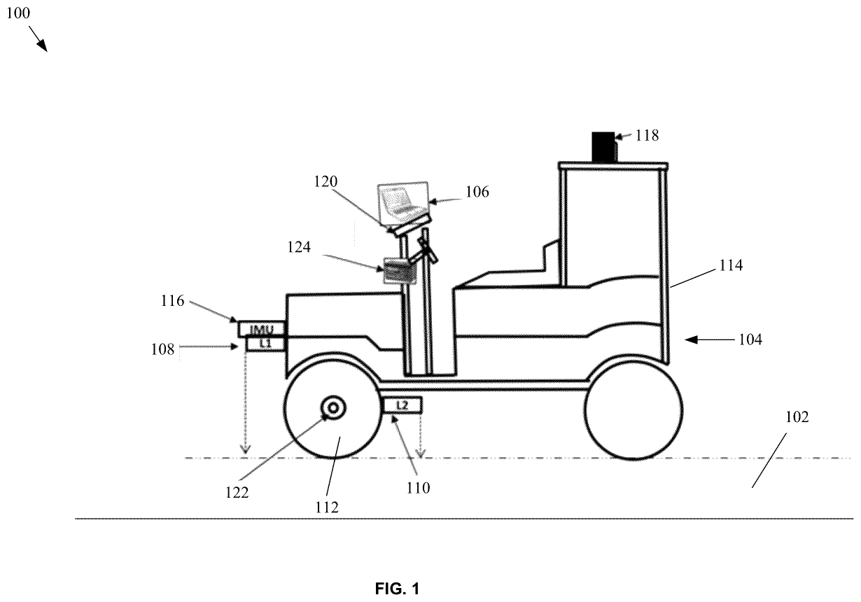

The motion/vibration sensor system may include one or more accelerometers 122, e.g., two accelerometer units, attached/fastened to the movable platform. The accelerometers 122 may be attached/fastened to two respective drums of the compactor 104 (e.g., roller) in order to measure motion/vibration of the respective drums, e.g., as shown in FIG. 1. The motion/vibration sensor system generate signals representing vibrations/movement of the movable platform (e.g., roller), including the rigid body movement of the movable platform.

The orientation/angle sensor may include an orientation unit 116, e.g., of an inertial measurement unit (IMU), attached/fastened to the movable platform, in particular to the rigid body 114. The orientation unit 116 may be attached/fastened to (including on top of) the first distance sensor 108 (e.g., LIDAR sensor) or elsewhere on the rigid body 114, e.g., fastened to the second distance sensor 110.

Described herein is a method/process 1100 (performed automatically by the system 100, also for estimating/measuring properties, including density, of a geomaterial layer 102 due to compaction by a compactor 104) that includes:
a. (continuously/continually/repeatedly) measuring a (surface) deformation Δ_{N} (or height change) of a geomaterial portion (of a construction area or the mining area) due to compaction of the geomaterial portion (including measuring during compaction of the construction area or the mining area);
b. (continuously/continually/repeatedly) receiving signals (from the distance sensor system 302) representing the measured deformation (“deformation signals”); and
c. (continuously/continually/repeatedly) automatically generating a (numerical) estimate/measure of a geomaterial layer property (e.g., density, stiffness, modulus, energy during compaction, or layer thickness) of the geomaterial portion based on:
   1. the measured deformation (including continuously/continually/repeatedly during the compaction of the construction area or the mining area); and
   2. a pre-defined relationship/model that associates (surface) deformation values with geomaterial layer property values (e.g., density values, stiffness values, modulus values, energy values, or layer thicknesses during compaction) e.g., including a correlation relationship/model and/or a trained machine-learning model.

The measuring of the deformation may be at a distance from/proximate to the geomaterial portion.

The method 1100 may include (continuously/continually/repeatedly) measuring motions/orientations of a movable platform synchronously with the measuring of the deformation.

The method 1100 may include (continuously/continually/repeatedly):
a. receiving signals (from the motion/orientation sensor system) representing the measured motions/orientations (“motion/orientation signals”), wherein the motion/orientation signals are synchronous with the deformation signals, and
b. automatically generating the estimate/measure of the geomaterial layer property of the geomaterial portion based on:
   i. the measured deformation;
   ii. a (numerical) correction based on the measured motion/orientations;
and
   iii. a pre-defined relationship/model that associates (surface) deformation values with geomaterial layer property values (e.g., density values, stiffness values, modulus values, energy values, or layer thicknesses during compaction) and

The method 1100 may include (continuously/continually/repeatedly) measuring/determining a geolocation (a location representing latitude and longitude in coordinates of the construction area or the mining area) of the geomaterial portion synchronously with the measuring of the deformation.

The method 1100 may include (continuously/continually/repeatedly):
a. measuring/determining the geolocation of the geomaterial portion so that the height of the geomaterial portion at same location can be measured after respective first and second compactions to generate at least a first measured height and a second measured height (thus the height at a location is measured after every pass, and height measured at each pass is subtracted from the previous pass to calculate the deformation from each pass);
b. receive signals (from the distance sensor system 302) representing the first and second measured heights of the geomaterial portion after respective first and second compactions, and
c. automatically generate a (numerical) estimate/measure of the geomaterial layer property (e.g., density, stiffness, modulus, energy during compaction, or layer thickness) of the geomaterial portion based on:
   i. the first and second measured heights (including determining/estimating the deformation from a numerical difference between the first and second measured heights); and
   ii. the pre-defined relationship/model representing a relationship between (surface) deformation values and geomaterial layer properties.

The method 1100 may include (continuously/continually/repeatedly):
a. generating a coded/color-coded map 500 of geomaterial layer property values of a plurality of geomaterial portions (and thus up to the entire construction area or the mining area) based on the geolocations and their respective geomaterial layer property values; and
b. displaying the coded/color-coded map 500 (to an operator of the compactor) to show when the compaction has reached a specified level (for the construction area or the mining area).

The measuring of the deformation method may include (continuously/continually/repeatedly):
a. automatically measuring a first height of the geomaterial portion before compaction (of the geomaterial portion);
b. automatically measuring a second height of the geomaterial portion after the compaction (of the geomaterial portion); and
c. determining the measured deformation from a difference between the measured first height and the measured second height.

The method 1100 may include generating signals representing vibrations/movement of the movable platform (e.g., roller), including the rigid body movement of the movable platform.

The method 1100 may include generating signals representing orientations of the movable platform (e.g., roller), including the rigid body movement of the movable platform.

### BRIEF DESCRIPTION OF THE DRAWINGS

Some embodiments of the present invention are hereinafter described with reference to the accompanying drawings in which:

a. FIG. 1 is a schematic diagram of a system with a compactor in the form of a roller (which may be referred to as an “instrumented roller”);

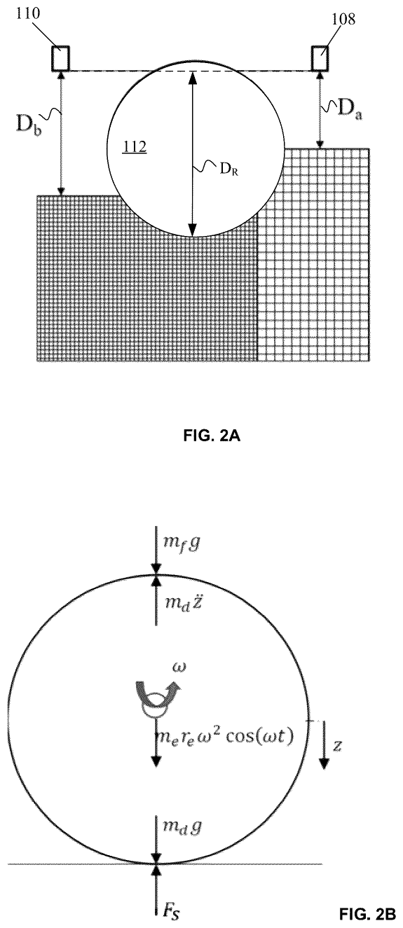

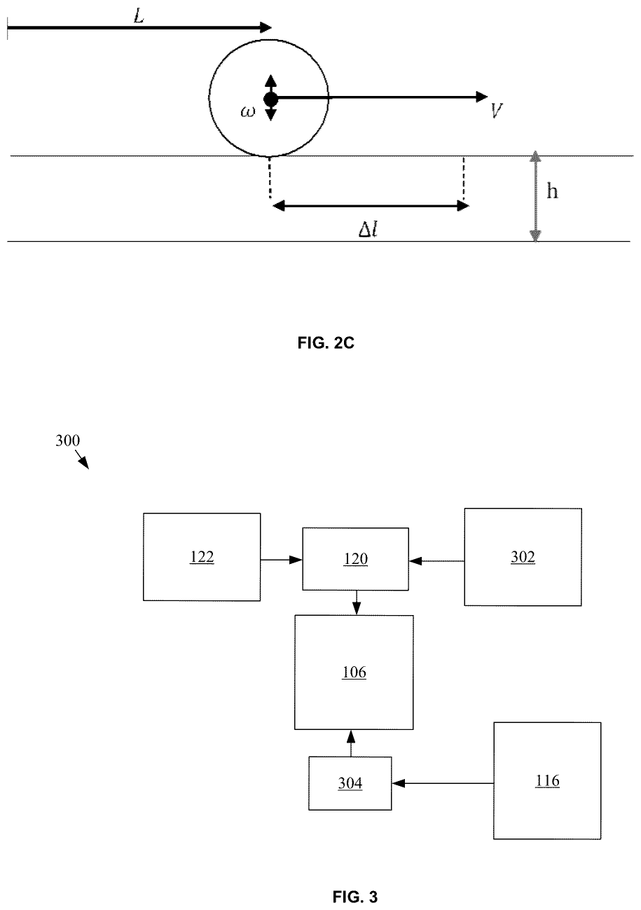

b. FIG. 2A is a side-view cross-sectional diagram of a compactor (roller) on a plurality of portions of geomaterial;

c. FIG. 2B is a simplified free body diagram for soil-drum interaction with values used to determine energy values during compaction;

d. FIG. 2C is a diagram of roller movement details with values used to determine energy values during compaction: FIG. 3 is a block diagram of sensors and a data acquisition component (DAQ) of the system;

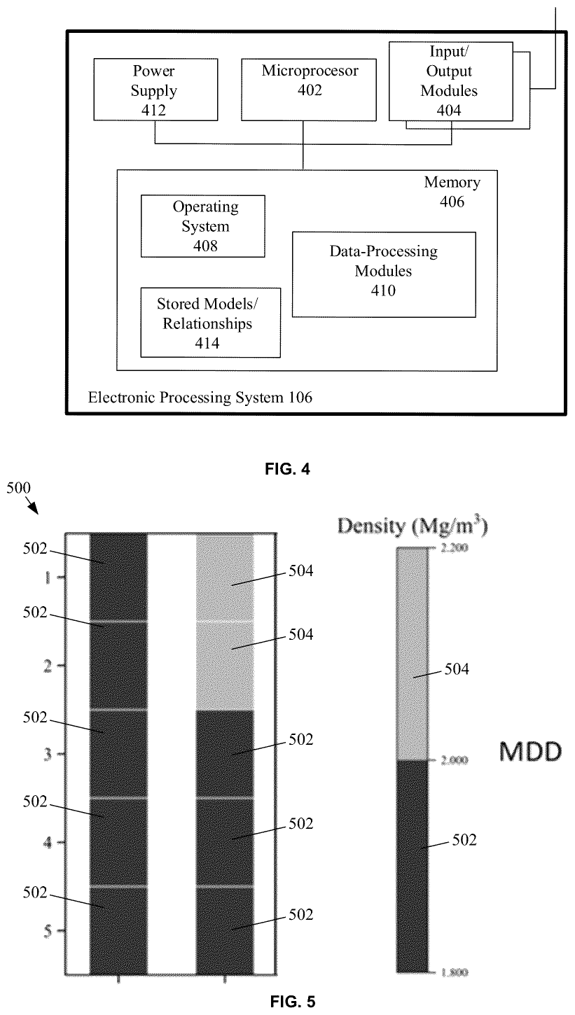

e. FIG. 4 is a block diagram of an electronic processing component of the system;

f. FIG. 5 is a sketch of a visible display of the system;

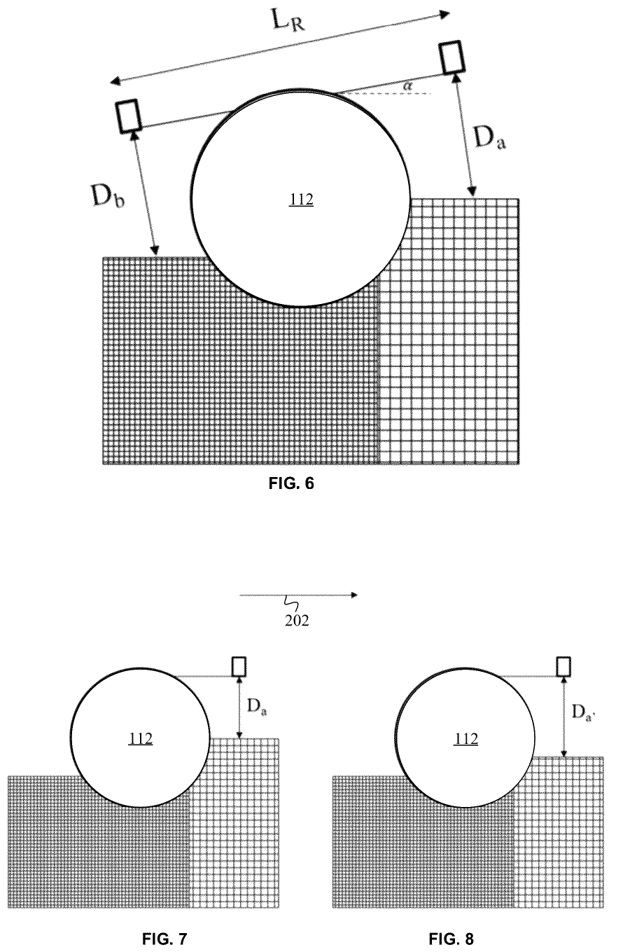

g. FIG. 6 is a side-view cross-sectional diagram of the compactor (roller) on an angle α to a surface of the plurality of portions of geomaterial;

h. FIG. 7 is a side-view cross-sectional diagram of the compactor (roller) on the plurality of portions of geomaterial during a first pass;

i. FIG. 8 is a side-view cross-sectional diagram of the compactor (roller) on the plurality of portions of geomaterial during a second pass;

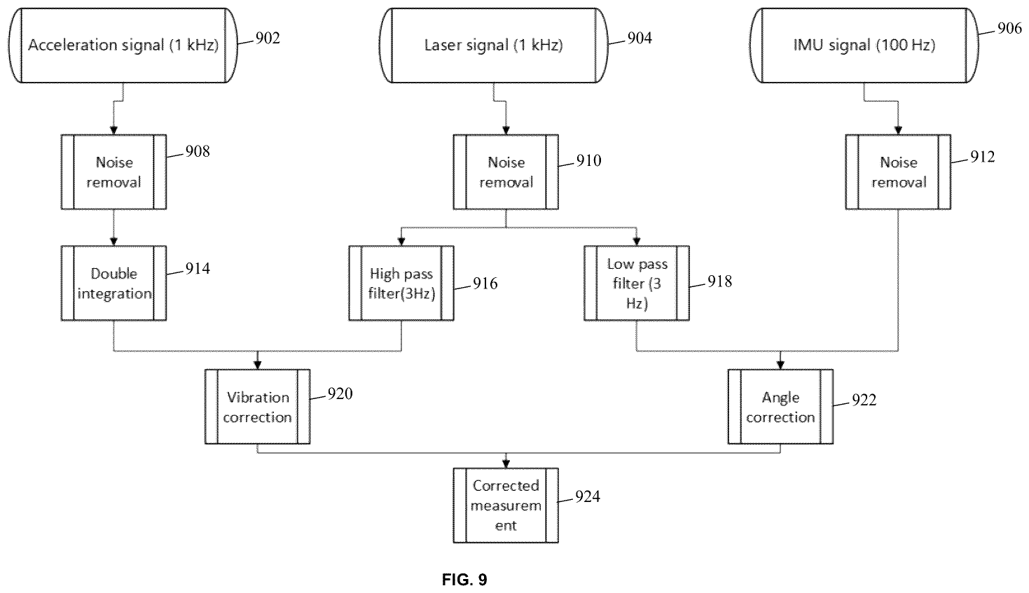

j. FIG. 9 is a flowchart of a signal correction method/process performed by the system;

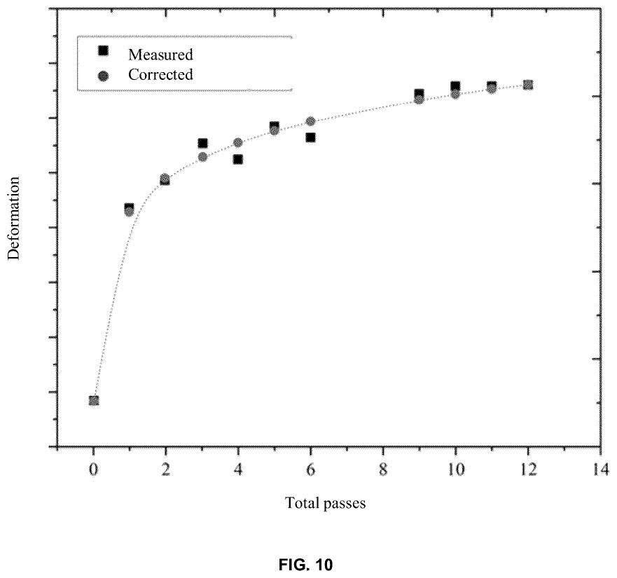

k. FIG. 10 is a graph of exemplary deformation measurements (Y axis) versus the number of compaction passes over a geomaterial (X axis) showing: (i) uncorrected measured datapoints (square) and (ii) corrected datapoints (circles) along a monotonic line;

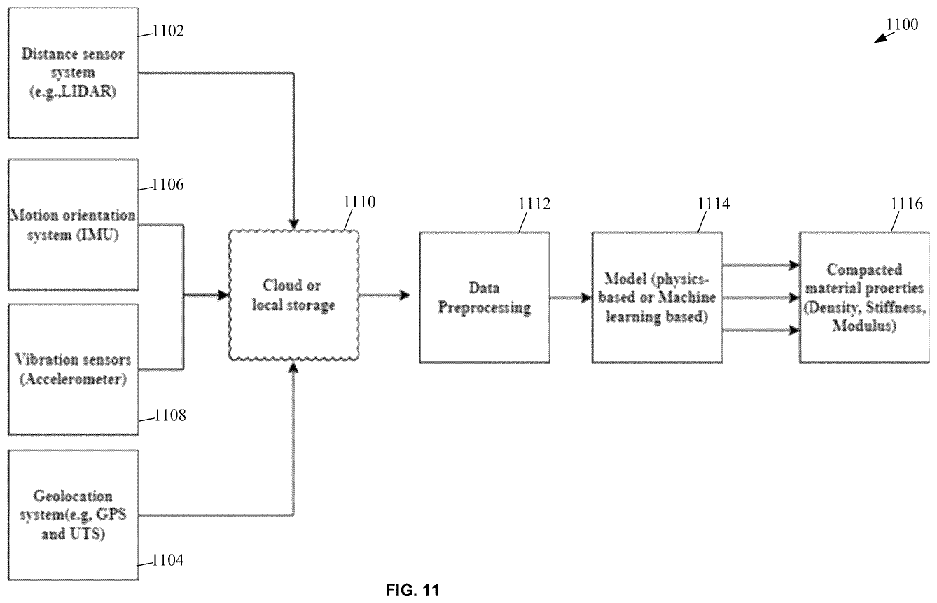

l. FIG. 11 is a flow chart of a method/process performed by the system;

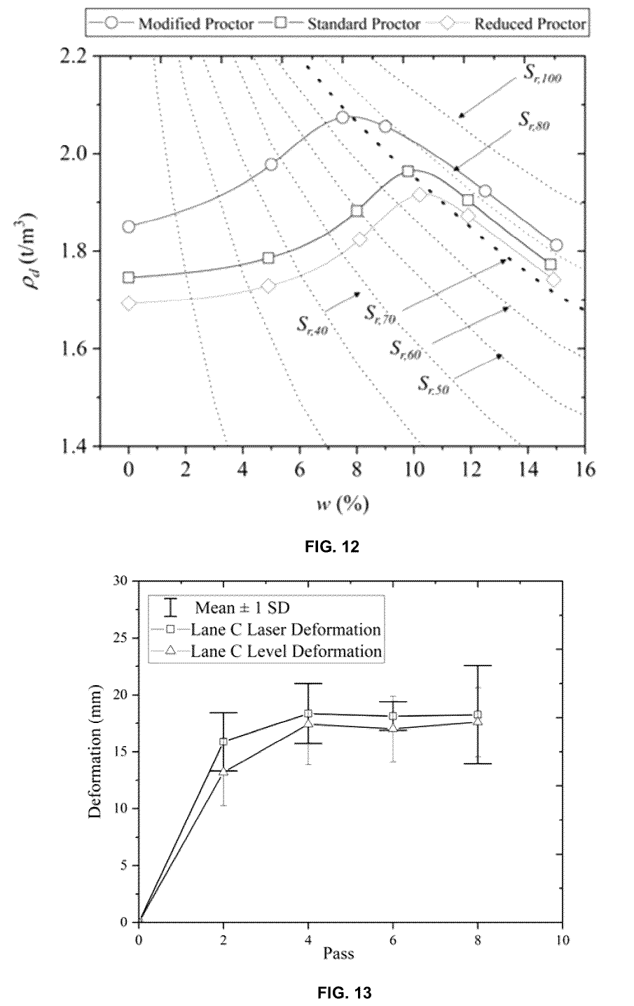

m. FIG. 12 is a graph of compaction curves of mutually different geomaterial types, including modified proctor (circles), standard proctor (square), reduced proctor (diamonds);

n. FIG. 13 is a graph of exemplary deformation (Y axis in mm, with error bars of 1 standard deviation) versus the number of compaction passes over a geomaterial (X axis) using: (i) the system and a laser distance sensor (squares); and (ii) point-by-point level and staff measurements (triangles);

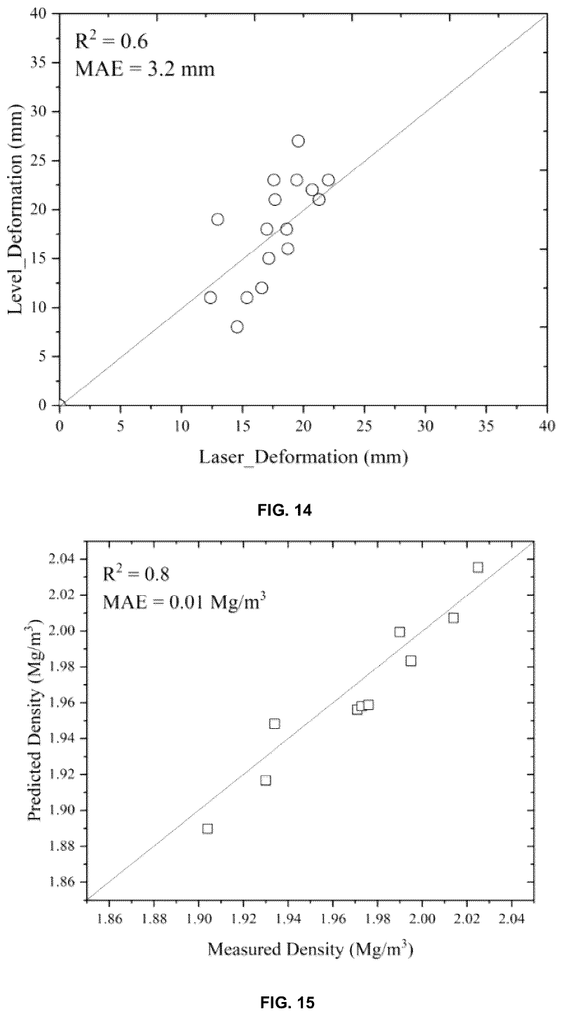

o. FIG. 14 is a graph of exemplary deformation measurements using a level and staff (Y axis in mm) versus deformation measurements using the system and the laser distance sensor (X axis in mm) for a plurality of example measurement points, with a line of linear fit (1:1 line); and

p. FIG. 15 is a graph of exemplary predicted density measurements using the system and the laser distance sensor (Y axis in Mg/m³) versus density measurements using NDG (X axis in Mg/m³) of the same geomaterial portion for each data point (square), with a line of linear fit (1:1 line).

### DETAILED DESCRIPTION

### System

As shown in FIG. 1, a system 100 (for estimating/measuring properties, including density, modulus, energy, stiffness and layer thickness, of a geomaterial layer 102 due to compaction by a compactor 104) may include:
a. a distance sensor system 302 located/oriented/configured to (continuously/continually/repeatedly) measure a (surface) deformation Δ_{N} (or height change) of a geomaterial portion (of a construction area or mining area) due to compaction of the geomaterial portion (including measuring during compaction of the construction area or the mining area); and
b. an electronic processing system 106 configured to (continuously/continually/repeatedly):
   i. receive signals from the distance sensor system 302 representing the measured deformation, and
   ii. automatically generate a (numerical) estimate/measure of a geomaterial layer property (e.g., density, stiffness, modulus, energy during compaction, or layer thickness) of the geomaterial portion based on:
      1. the measured deformation (including continuously/continually/repeatedly during the compaction of the construction area or the mining area); and
      2. a pre-defined relationship/model that associates (surface) deformation values with geomaterial layer property values (e.g., density values, stiffness values, modulus values, energy values during compaction, or layer thicknesses), e.g., a correlation relationship/model or a trained machine-learning model.

The geomaterial may include a plurality of geomaterial types, including: granular materials (e.g., sand, crushed rocks or unbound granular material (UGM)), non-granular materials (e.g., clay, silt), particulate materials (e.g., construction and demolition waste, any particulate synthetic material or mixtures), and/or asphalt/asphalt-concrete (e.g., of a surface course (top layer) of a road). The geomaterial may be in or of a road, and/or a subgrade (or compacted subgrade) of a road or engineered earthworks in civil construction, or in a mine site, including in a surface mine (e.g., in a bench) or in an underground mine (e.g., a tunnel). The compactor 104 may include a landfill compactor, a multi-wheel roller, a pneumatic tyre roller, a plate compactor, a static roller compactor, a vibrating compactor, a pad foot roller, a paver, a loaded vehicle, or a slash presser roller.

The distance sensor system 302 is configured/mounted/arranged to measure the deformation without touching the geomaterial (thus “remotely”, “proximally”, “from a distance”), so this system 100 may address the problem of requiring destructive/invasive measurements with pre-existing technologies, and may allow continuous monitoring of the geomaterial layer properties. This system 100 may address the problem of pointwise geomaterial layer property/density measurements being slow to make with pre-existing technologies by allowing for the measurements to be made continuously during compaction since the distance sensor system 302 does not interfere with the compactors, e.g., this system 100 can measure the geomaterial layer properties/density of road compaction during the compaction, and this may be referred to as “live” or “real-time” monitoring of the geomaterial layer properties/density during compaction.

The geomaterial layer properties may include the density, a layer stiffness, a layer modulus, energy imparted by the compactor, and/or a layer thickness during compaction. The geomaterial layer property values may include: values of stiffness and/or modulus during compaction, or the layer thickness, which may be alternative QA criteria to density values; and/or the energy imparted by the compactor 104.

The distance sensor system 302 may include:
a. at least one first distance sensor 108 (also referred to as a “first sensor arrangement”) located/oriented/configured to measure a first height of the geomaterial portion before compaction (of the geomaterial portion); and
b. at least one second distance sensor 110 (also referred to as a “second sensor arrangement”) located/oriented/configured to measure a second height of the geomaterial portion after compaction (of the geomaterial portion).

The distance sensor system 302 or the electronic processing system 106 may be configured to determine the measured deformation from a difference between the measured first height and the measured second height. The distance sensor system 302 may include a plurality of distance sensors (including the first distance sensor 108 and the second distance sensor 110) in the form of laser systems, e.g., Light Detection and Ranging (LIDAR) sensors or triangulation laser sensors.

As shown in FIG. 1, the first distance sensor 108 may be configured for attachment to a first side (in front) of a compactor element 112 (e.g., the roller), with the second distance sensor 110 configured for attachment to a second side of (behind) the compactor element 112, such that the first distance sensor 108 can measure the first height before the compactor element 112 operates to compact the geomaterial portion (by moving across the geomaterial portion) and the second distance sensor 110 can measure the second height after the compactor element 112 operates to compact the geomaterial portion. This configuration of the distance sensor system 302 may address a problem of the compactor element 112 interfering with the sensor measurements, e.g., by blocking the sensors.

The laser systems of the distance sensors 108,110 are mounted/arranged/attached/fastened on/to a rigid body 114 of a movable platform (which can be the compactor 104) and oriented to face toward the geomaterial layer 102 such that the geomaterial portion is substantially at a focal distance of each of the laser systems. Having the geomaterial portion substantially at the focal distance means a spot size of the laser system on the geomaterial portion is substantially at its optimal size (which may depend on geomaterial type), thus the accuracy of the measured distance/height is substantially highest.

As shown in FIG. 2, the compactor 104 (e.g., the compactor element 112) is configured to move over a plurality of portions of the geomaterial in a selected direction 202. As shown in FIG. 2, the distance sensor system 302 can include at least one range sensor, e.g., at least one first range sensor 204 at the front of the compactor element 112 and at least one second range sensor 206 at the rear of the compactor element 112, configured for measuring the deformation during the compaction as D_{b}−Dₐ in a single pass. The first range sensor can include the at least one first distance sensor 108 (which may include a laser, e.g., a first LIDAR sensor or triangulation laser sensor) configured to measure a first range Dₐ, and the second range sensor can include the at least one second distance sensor 110 (e.g., a second LIDAR sensor or triangulation laser sensor) configured to measure a second range D_{b}, and the measured deformation may be determined from the difference between the measured first height and the measured second height, thus from a difference between the first range Dₐ and the second range D_{b}, e.g., deformation (Δ_{N})=D_{b}−Dₐ.

The pre-defined relationship/model is accessed by the electronic processing system 106 that forms an electronic processing component of the system 100. The pre-defined relationship/model may include a trained machine-learning (ML) model, e.g., an artificial neural network (ANN), configured to automatically generate the estimate/measure of the geomaterial layer property/density values based on the measured deformation. The pre-defined relationship/model may include a correlation relationship/model, e.g., a relationship based at least in part on an analytical model. The pre-defined relationship/model may be or include a constitutive relationship/model that associates the deformation values with the geomaterial layer property/density values based on the geomaterial type (thus geomaterial type is an input to the model/relationship).

As shown in FIGS. 1 and 3, the system 100 includes:
a. a distance sensor system 302 located/oriented/configured to (continuously/continually/repeatedly) measure a (surface) deformation Δ (height change) of a geomaterial portion (of a construction area or a mining area) due to compaction (of the geomaterial portion);
b. a motion/orientation sensor system located on the movable platform and mounted/oriented/configured to (continuously/continually/repeatedly) measure motions/orientations of the movable platform when the movable platform is moving (including on the rigid body 114 of the movable platform) synchronously with the measurements of the distance sensor system 302:
c. an electronic processing system 106 configured to (continuously/continually/repeatedly):
   i. receive signals from the distance sensor system 302 representing the measured deformation (“deformation signals”) and from the motion/orientation sensor system representing the measured motions/orientations (“motion/orientation signals”), and
   ii. automatically generate a (numerical) estimate/measure of a geomaterial layer property (e.g., density, stiffness, modulus during compaction, or layer thickness) of the geomaterial portion based on:
      1. the measured deformation;
      2. a (numerical) correction based on the measured motion/orientations; and
      3. a pre-defined relationship/model that associates (surface) deformation values with geomaterial layer property values (e.g., density, stiffness, modulus during compaction, or layer thickness); and
d. a geolocation unit 118 (or “positioning system”) located/mounted/oriented/configured to (continuously/continually/repeatedly) measure/determine a geolocation (representing latitude and longitude in coordinates of the construction area or the mining area) of the geomaterial portion (to generate a coded/color-coded map of geomaterial layer property/density values of the (entire) construction area or mining area).

This system 100 may address a problem of accurately estimating QA-relevant layer properties, e.g., density, stiffness, modulus during compaction and/or later thickness, of the geomaterial portion based on measurements from the movable platform because measuring small deformations/height changes can be difficult and insufficiently accurate without vibration/angle correction.

The electronic processing system 106 may be configured to (continuously/continually/repeatedly): generate a coded/color-coded map of geomaterial layer property/density values of the (entire) construction/mining area based on the geolocations and their respective geomaterial layer property values.

The movable platform may include or be in the form of the compactor 104.

The motion/orientation sensor system may include a motion/vibration sensor system and/or an orientation/angle sensor system.

The motion/vibration sensor system may include one or more accelerometers 122, e.g., two accelerometer units. The accelerometers 122 may be attached/fastened to two respective drums of the compactor 104 (e.g., roller) in order to measure motion/vibration of the respective drums, e.g., as shown in FIG. 1. The motion/vibration sensor system generate signals representing vibrations/movement of the movable platform (e.g., roller apparatus), including the rigid body movement of the movable platform.

The orientation/angle sensor may include an orientation unit 116, e.g., of an inertial measurement unit (IMU), attached/fastened to the movable platform, in particular to the rigid body 114. The orientation unit 116 may be attached/fastened to (including on top of) one of the at least one first distance sensor 108 (e.g., LIDAR sensor), e.g., as shown in FIG. 1, or elsewhere on the rigid body 114, e.g., fastened to the second distance sensor 110.

The system 100 can be retrofitted to include the compactor 104, e.g., a roller, using a fastener system, e.g., including a bracket and one or more mechanical fasteners (e.g., screws), depending on the size and shape of the compactor's rigid body, configured to hold the distance sensor system 302, and the motion/orientation sensor system, to the compactor 104 or the movable platform.

The electronic processing system 106 may be mounted on/to the compactor, and/or may include a wireless communications unit, e.g., with a RF transmitter, e.g., a WiFi or Bluetooth transmitter, configured to receive the signals wirelessly from the distance sensor system 302, and the motion/orientation sensor system and/or the geolocation unit 118.

### Electronic System

As shown in FIGS. 1 and 3, the system 100 includes a data acquisition component (DAQ) 120 connected to: the distance sensor system 302 to receive the deformation signals; and the electronic processing system 106 to send signals as data. The DAQ 120 may include a 16-bit, 250 kHz DAQ from National Instruments (NI).

Each laser system may include a triangulation displacement laser line sensor. The triangulation displacement laser line sensor transmits a beam of light to the object to be measured, and the reflected light strikes the receiver line in the detector at a unique angle. Depending on the angle of incidence, the distance to the object is calculated. The light source for the laser may be a pulsed red laser diode, e.g., with a wavelength of 600 nm. Details for an example laser system are provided in Appendix A. The example laser system may be able to measure with high precision and high accuracy in a vibrating environment. The beam of the laser may be Class 2, which makes it safer to use.

The orientation unit 116 may have six degrees of freedom, provided by a triaxial acceleration sensor and a triaxial gyroscope that provide acceleration, inclination and rotation rate. A fusion algorithm, provided in the orientation unit 116 (thus “inbuilt”) may provide compensation for external acceleration disturbance: the inbuilt fusion algorithm removes errors due to the vibration of the compactor 104 in use. The orientation unit 116 may provide reliable measurements even in a noisy environment. The IMU outputs eight elements (acceleration, angular rate, rotational acceleration, gravity vector, linear acceleration, rotation angles, quaternion and temperature). The rotation angle data from the orientation unit 116 (e.g., IMU) may be used by the electronic processing system 106 for the inclination correction and the roll correction described hereinbefore.

As shown in FIG. 3, the system 100 may include a Controller Area Network (CAN) bus 304 to connect the orientation unit 116 to the electronic processing system 106, e.g., an integrated CAN SAE J1939 interface and a CAN-to-USB adaptor to send the data to the electronic processing system 106. The CAN bus 304 allows the data to be recorded by just two wires.

The one or more accelerometers 122 are configured/arranged/positioned/oriented to track the movable platform's rigid body movement and measure vibration being transferred from the compacting element (e.g., the drum) to the distance sensor system. Example maximum drum acceleration amplitudes observed during testing were ±8 g (gravitational force). The example accelerometer specifications other than the measurement range included high sensitivity, high sampling frequency, and low-temperature error. An example accelerometer included a Brüel & Kjær Miniature Triaxial Piezoelectric Constant Current Line Drive (CCLD) Accelerometer with TEDS. To mount the accelerometers 122, the system 100 may include respective fasteners, e.g., including polycarbonate mounting clips.

The system 100 may include coaxial cables arranged to connect the accelerometers 122 to the DAQ 120 and the electronic processing system 106. The coaxial cables may include radio-frequency connectors, e.g., BNC connectors (“Bayonet Neill-Concelman” connectors) for connection to the accelerometers 122 and the DAQ 120.

The system 100 may include the geolocation unit 118 configured to connect to a geolocation system (e.g., a GNSS/GPS unit/module and/or survey/ranging unit/module) for measuring respective locations of the height/distance/deformation measurements. The geolocation unit 188 can include: a ground unit/module for a global navigation satellite system (GNSS) or a Global Positioning System (GPS), e.g., for outdoor operation; and/or a survey/ranging unit/module that may include a total station (TS) or total station theodolite (TST) or universal total station (UTS), e.g., for indoor/underground operation.

As shown in FIG. 4, the electronic processing system 106 may include a microprocessor 402 connected to input/output modules 404, and machine-readable memory 406 connected to the microprocessor 402. The input/output modules 404 are connected to the distance sensor system 302 (e.g., via the DAQ 120), to the motion/orientation sensor system (e.g., including via the DAQ 120 for the accelerometers 122 and via the CAN bus 304 for the orientation unit 116). The input/output modules 404 may be configured to connect to a remote/cloud server, e.g., via a wireless network and the Internet (e.g., WiFi) to access the pre-defined relationship/model. The memory 406 includes an operating system 408, e.g., MS Windows, and the machine-readable data-processing modules 410 that control the microprocessor 402 to perform the data-processing methods described herein. The electronic processing system 106 may include a power supply 412, e.g., a battery and/or power plug connected to a power source 124 of the compactor 104. The machine-readable data-processing modules 410 may include scripts and modules written using National Instruments' Laboratory Virtual Instrument Engineering Workbench (LabVIEW). The machine-readable modules 410 control the microprocessor 402 to acquire the signals, and to perform the real-time signal analysis described herein: e.g., the machine-readable modules 410 include a model module that includes or accesses the pre-defined relationship/model (e.g., from stored models/relationships 414 in the memory 406, or from the remote/cloud server), and that controls the microprocessor 402 to correlate or convert the measured deformation values to the estimated geomaterial layer property/density values.

The data-processing modules 410 may be configured to control the microprocessor 402 to determine/calculate/estimate the geomaterial layer property values based on the measured deformation values for the geomaterial portion and using a selected corresponding one of the pre-defined relationships.

The data-processing modules 410 may be configured to control the microprocessor 402 to determine/calculate/estimate these geomaterial layer property values directly from the measured deformation/heights using respective pre-defined relationships/models that associate the (surface) deformation values with the geomaterial layer property values.

The determined/calculated/estimated geomaterial layer property values may be displayed in real time, e.g., to an operator of the compactor, e.g., to determine when compaction has reached a specified level for the construction/mining area. To this end, the data-processing modules 410 may include a display module configured to generate the coded/color-coded map 500 of the geomaterial layer property values of the construction/mining area, e.g., as shown in FIG. 5. The electronic processing system 106 may include an audio/visual component (“AV component”, e.g., a speaker and/or visible display) that is controlled by the display module to display the coded/color-coded map 500 of the area based on the measured/estimated geomaterial layer property values for the area, e.g., including respective measured/estimated geomaterial layer property/density values for portions of the area. The map 500 may display whether the respective measured/estimated geomaterial layer property values are above or below one of the threshold geomaterial layer property values, thus visually indicating whether the measured/estimated values for the portions are sufficient for the pre-defined QA specification, and thus whether further compaction is required. As shown in FIG. 5, the portions may be represented by substantially rectangular boxes, e.g., 1 metre in length with a width substantially equal to the compactor width, and the map may include red boxes 502 for low density (below the threshold, e.g., 2,000 Mg/m³) and green boxes 504 for high density (above the threshold).

### Alerting

The machine-readable modules 410 may include an alerting module that is configured to automatically compare the measured/estimated geomaterial layer property values (which includes a plurality of values for the area) with one or more pre-selected threshold values of the geomaterial layer properties (e.g., pre-defined by a density specification for the geomaterial portion and the construction/mining area, e.g., in QA documentation/data), and to generate an alert/flag/indicator signal if the measured/estimated geomaterial layer property values are more, less and/or equal to the threshold values. The system 100 may thus address a problem of applying too little or too much compaction by indicating when the pre-defined density has been reached, e.g., for a road project, thus in “real time” while the compactor is still being operated. The AV component may be controlled by the alerting module to play audible and/or visible signals in response to the alert/flag/indicator signal.

### Corrections

The machine-readable modules include a correction module that performs the signal correction method/process to determine and apply correction(s) to improve accuracy of the determined/calculated/estimated geomaterial layer property values.

The corrections can include a correction for the measured motion/orientations in the form of an inclination correction (due to rotation around an axis substantially parallel to the construction/mining area and substantially perpendicular to a movement/travel direction of the moving platform), including correction for inclination of the distance sensor system relative to the geomaterial portion, e.g., due to inclination of the moving platform (e.g., vehicle) relative to the construction/mining area (e.g., road). The measurement error from instantaneous inclination of the distance sensor system (e.g., due to a compactor element 112 in the form of a roller 112) is corrected by measuring the inclination/pitch (a) of the distance sensor system relative to the geomaterial portion using the orientation unit 116. The inclination correction may include adjusting the measured deformation based on the measured inclination/pitch (a), including based on a trigonometric function applied to the measured inclination/pitch (a), and on a mutual separation L_{R} in the movement/travel direction between the first distance sensor and the second distance sensor (in a direction substantially perpendicular to a measurement direction of the first distance sensor and the second distance sensor), e.g., as shown in FIG. 6. Including the inclination correction, the corrected deformation may be determined according to the following pre-defined relationship:

$${Δ}_{N} = ( {D}_{b} - {D}_{a} ) cos ( α ) + {L}_{R} sin ( α )$$

In an example where the L_{R} is 1 meter, an inclination of 1 degree changes the uncorrected deformation by around 17 mm.

The correction for the measured motion/orientations may include a roll correction (due to rotation around an axis substantially parallel to the movement/travel direction of the moving platform), including correction for a rocking motion (represented by roll angle (B)) of the moving platform. The roll angle (B) can be measured with the same orientation unit that measures the inclination/pitch (a). The roll correction may include adjusting the measured deformation based on the measured roll angle (B), including based on a trigonometric function applied to the measured roll angle (B). Including the roll correction, the corrected deformation may be determined according to the following pre-defined relationship:

$${Δ}_{N} = ( {D}_{b} - {D}_{a} ) cos ( β )$$

Including the inclination correction and the roll correction, the corrected deformation may be determined according to the following pre-defined relationship:

$${Δ}_{N} = ( {D}_{b} - {D}_{a} ) cos ( α ) cos ( β ) + {L}_{R} sin ( α )$$

The correction (also referred to as a “correction factor”) may be based on electrical noise removal to improve the measurement accuracy, including pre-processing to mitigate electrical noise from the distance sensor system and/or the motion/orientation sensor system (e.g., the measurements of inclination/pitch (a) are de-noised), which may include one or more of:
a. singularity removal (including removal of sudden shifts or abrupt changes in the signal by identifying points where the mean value of the signal is changing abruptly, above a selected threshold, and replacing it with the mean of a plurality of selected neighborhood points);
b. detrending (including removing effects of a trend to only show differences in values from the trend); and
c. frequency filtering, including band-pass filtering (including removing very low and very high frequency noise from the signal).

The correction for the measured motion/orientations may include vibration noise removal in the form of correction for vibration in the measurements of the distance sensor system (e.g., laser data) using the measured motions, which may be from the accelerometers 122 (e.g., in accelerometer data). In implementations, the rigid body 114 may vibrate, and so the distance data may contain the noise due to the vibration of the compactor: even if noise vibrations of the compactor are of the order of a sub-mm, they may mitigate the accuracy of the deformation measurements/calculations. To remove the vibration noise from the distance data, the sensor data from the distance sensor system (e.g., from both distance/range sensors) may be pre-processed to correct for the vibrations/movement. The displacement noise may be calculated from an acceleration signal by double integrating the acceleration noise, and the system (generally the electronic processing system) is configured to remove the displacement noise from the distance measurements.

In experimental embodiments, the distance sensor system (e.g., the laser systems) may be unable to measure signals less than a lower frequency (depending on the geomaterial type, e.g., 0.1 Hz), and error due to the inclination may be around a determined frequency (depending on the geomaterial type, e.g., 3 Hz). The lower frequency and the determined frequency may be determined experimentally for the geomaterial type. Therefore, the system may include a plurality of frequency filters (including bandwidth filters) to separate the signals from the distance sensor system for separate correction of the motion (vibrations) and the orientation (inclination), e.g., as shown in FIG. 9.

The system 100 is configured to merge the separate corrections to yield the corrected measured distance.

### Pre-Defined Relationship/Model

The pre-defined relationship/model correlates or converts the deformation values to the geomaterial layer property/density values.

The pre-defined relationship/model may include the pre-defined relationships described hereinbefore, including the pre-defined relationships for the stiffness values, the layer thickness values, the modulus values, the energy values, and/or the density values.

For the stiffness values, three different deformations can be calculated/generated/estimated/measured using at least one pre-defined relationship representing differences between two of the first range Dₐ, the second range D_{b} and the initial distance of the sensor from the ground D_{R}, which is a constant value shown in FIG. 2, e.g., the following pre-defined relationships:
a. plastic deformation or deformation (Δ_{N})=D_{b}−Dₐ:
b. total deformation (Δ_{N,total})=D_{R}−Dₐ; and
c. elastic deformation (Δ_{N,elastic})=D_{R}−D_{b}.

To calculate/generate/estimate/measure two types of stiffness values (K_{N}) of the material at a particular pass N, the pre-defined relationship/model may include at least one pre-defined relationship representing a force F applied due to compactor (which includes static and vibratory load), and the total and elastic deformation Δ_{N,total}, Δ_{N,elastic} during pass N, e.g., the following pre-defined relationships:

$${K}_{N , secant} = \frac{F}{{Δ}_{N , total}}$$

$${K}_{N , elastic} = \frac{F}{{Δ}_{N , elastic}}$$

To calculate/generate/estimate/measure the layer thickness values, e.g., after a particular pass N (H_{N}), the pre-defined relationship/model may include at least one pre-defined relationship representing an initial layer thickness value Hᵢ and a total deformation/compaction value until pass N (ΔH_{N}), e.g., using the following relationship:

$${H}_{N} = {H}_{i} - Δ {H}_{N} ,$$

and representing the total deformation/compaction value until pass N (ΔH_{N}) calculated/generated/estimated using a summation of all the deformation values until pass N, e.g., the following pre-defined relationship:

$$Δ {H}_{N} = {\sum }_{i = 1}^{N} {Δ}_{N}$$

To calculate/generate/estimate/measure the modulus values, e.g., the two moduli of the material at a particular pass N (M_{N}), the pre-defined relationship/model may include at least one pre-defined relationship representing: a stress applied due to compactor (σ_{Z}) (which is due to static and vibratory load and should be found using appropriate relationships from force); and the total and elastic deformation during pass N (Δ_{N,total}, Δ_{N,elastic}), e.g., the following pre-defined relationships:

$${M}_{N , secant} = \frac{{σ}_{Z}}{( \frac{{Δ}_{N , total}}{{H}_{N}} )}$$

$${M}_{N , elastic} = \frac{{σ}_{Z}}{( \frac{{Δ}_{N , elastic}}{{H}_{N}} )}$$

Alternatively/additionally, the pre-defined relationship/model may include at least one pre-defined relationship representing the stiffness values and a pre-defined numerical model.

To calculate/generate/estimate/measure the energy values, e.g., the total energy imparted (Eₜₒₜₐₗ), the pre-defined relationship/model may include at least one pre-defined relationship representing a plurality of energy contributions E₁, E₂, E₃, e.g., the following pre-defined relationship:

$${E}_{total} = {E}_{1} + {E}_{2} + {E}_{3} .$$

The at least one pre-defined relationship for the plurality of energy contributions E₁, E₂, E₃ may include relationships with one or more of the following calculated/generated/estimated/measured values of or relating to the roller:
a. F_{S}=soil-drum interaction force (N);
b. m_{d}=drum mass (kg);
c. m_{f}=frame mass (kg);
d. m=total mass (m_{f}+m_{d}) (kg);
e. ω=2πf=circular vibration frequency (rad/s);
f. f=frequency of excitation (Hz);
g. z=displacement of drum (m);
h. \ddot{z}=acceleration of drum (m/s²);
i. mₑrₑ=eccentric moment of unbalanced mass (kg-m);
j. t=time(s);
k. g=gravitational acceleration (9.81 m/s²);
l. V=linear velocity (m/s);
m. Δl=travel width (m);
n. r-radius of the drum (m);
o. I=Moment of inertia ½mr² (kg-m²); and
p. Δ_{N}=change in layer thickness (deformation).

The at least one pre-defined relationship for the plurality of energy contributions E₁, E₂, E₃ may include:

$${E}_{1} = ( \frac{\frac{1}{2} {IV}^{2}}{{r}^{2}} + \frac{1}{2} m {V}^{2} ) \times \frac{Δ l}{2 π r} + m g {Δ}_{N} f \frac{Δ l}{V}$$

$${E}_{2} = ( m g + \frac{1}{2} {m}_{e} {r}_{e} {ω}^{2} ) \times zf \frac{Δ l}{V}$$

$${E}_{3} = ( \frac{1}{2} {m}_{e} {r}_{e} {ω}^{2} ) \times {Δ}_{N} f \frac{Δ l}{V}$$

To calculate/generate/estimate/measure the density values, the pre-defined relationship/model may include at least one one-dimensional (1D) compression relationship configured to automatically generate the estimate/measure of the density based on the measured deformation in 1D (with no calibration or training required). The geomaterial portion may be assumed to be deforming only vertically in a 1D compression, and thus the final density (ρ_{f}) after compaction can be estimated/calculated using the 1D compression relationship, e.g., the following relationship, which is based on the measured total deformation ΔH_{N}, an initial test layer thickness Hᵢ (e.g., measured using an optical level and staff, or a universal total station (UTS)), an initial test layer density ρᵢ (measured at one or more places using an in-situ technique, e.g., a nuclear density gauge (NDG) or a sand cone test) and Δ_{N} representing the deformation during pass N:

$$\frac{Δ {H}_{N}}{{H}_{i}} = \frac{{ρ}_{f} - {ρ}_{i}}{{ρ}_{f}}$$

$$Δ {H}_{N} = {\sum }_{i = 1}^{N} {Δ}_{N} .$$

The pre-defined relationship/model may include a two-dimensional or three-dimensional (2D/3D) compression relationship configured to automatically generate the estimate/measure of the density based on the measured deformation (with no calibration or training required, and potentially a higher accuracy than the 1D relationship, at least for some implementations). The 2D/3D compression relationship includes the 1D relationship and correction factor CF, i.e., ρ_{f2D}=CF×ρ_{f1D}. The correction factor CF is measured/determined experimentally using for each material type and compactor in the test strip/correlation strip (e.g., one roller width and around 10 m in length), and can include a linear function of the ratio between the total deformation/compaction value until pass N, ΔH_{N} (which is the measured test deformation), and the measured initial test layer thickness Hᵢ, or ΔH_{N}/Hᵢ, for example:

$$CF = \frac{Δ {H}_{N}}{{H}_{i}} a + b$$

where a and b are values from a linear fit. The test measurements are used to fit (e.g., by linear regression) the linear function CF to the test measurements of density and deformation (the measurements being made using an in-situ technique), thus to determine a and b for the relevant material type and compactor.

The CF may be determined/estimated from prior experiment measurements with mutually different geomaterial types and different compactors. The CF may be modelled using a trained ML model that has been trained using the following inputs and outputs: (i) the inputs include deformation measurements, compactor weight and geomaterial type; and (ii) the outputs include the CF. The system then uses the trained ML model of the CF to provide/estimate the CF values by inputting the material properties and compactor weight.

The pre-defined relationship/model may include the trained ML model, which may include an artificial neural network (ANN) and/or a Random Forest (RF). The ML model may be trained using the following inputs and outputs: (i) the inputs may include deformation measurements, compactor weight and geomaterial type; and (ii) the outputs may include the geomaterial layer property/density estimates/values. The ML model is first trained, then used for prediction/estimation of the geomaterial layer property/density. If the ML model depends on the compactor weight and the geomaterial type, the ML model may need to be trained for each compactor weight (of the compactor) and each geomaterial type (of the geomaterial portion). The training of the ML model can include, before a large area of compaction is planned, using a test strip/correlation strip (e.g., one roller width and around 10 m in length) to measure the input deformation values and the output geomaterial layer property/density values using an in-situ technique, e.g., a nuclear density gauge (NDG) or a sand cone test. Sometimes, the operators/contractors are not interested in knowing the exact value of geomaterial layer property/density, but just want to know whether the area is dense or loose: in that case, the ML model is a classification ML instead of a regression ML model, and with the classification ML model: (i) the inputs may include the deformation measurements, the compactor weight and the geomaterial type; and (ii) the outputs may include two more classes, e.g., “dense” or “loose”/“green” or “red”, e.g., based on at least one selected threshold value of the geomaterial layer property/density from the QA specification for the area.

The measured test deformation ΔH_{N}, measured in the field, may contain noise because of uncertainties involved with testing, measurement, equipment limitations, and human error. Therefore, the raw value of the ΔH_{N} may be de-noised using ML techniques to smoothen the behaviour. For example, a measured deformation pattern with the number of passes may not be monotonically increasing, as shown by the square datapoints in FIG. 10, which may be de-noised using ML methods described hereinafter, and the de-noised values may be used instead the measured raw values data points to calculate/estimate the density. The ML model that correlates or converts the deformation values to the geomaterial layer property/density values can be modified and informed about the noise using a restriction relationship/equation as an input to the training. The restriction relationship/equation represents ΔH_{N} increasing monotonically; or in other words, with an increase in total passes (N), ΔH_{N} always increases, which can be written as ΔH_{N+1}−ΔH_{N}>0. The regular loss function of the ML model may therefore be modified by adding denoising loss function L_{DN}. The denoising loss function depends on a difference of the predicted deformation as a pair, which may be calculated as

$${J}_{N} = Δ {H}_{N} - Δ {H}_{N + 1}$$

To enforce the restriction relationship/equation, any positive value of J_{N} is defined as noise in the measurement, and thus LON may be calculated as a non-zero occurrence of a Rectified Linear Unit of the difference of the predicted deformation, ReLU(J_{N}), summed over all the cycles, then multiplied by a suitable hyperparameter Δ_{DN}, which is decided using trial and error, e.g.,

$${L}_{DN} = {λ}_{DN} {\sum }_{N = 1}^{N} ReLU ( {J}_{N} ) .$$

If the value of Hᵢ cannot be estimated, e.g., due to the unavailability of geolocation from the geolocation signal, it can be approximated using a test deformation-to-thickness relationship, as follows, which the electronic processing system uses to determine/estimate/calculate the initial test layer thickness Hᵢ based on a plurality of model parameters (C₂ and m), a number of cycles (N) and the stress applied due to the compactor (σ_{Z}), e.g.:

$$Δ {H}_{N} = {H}_{i} ln ( 1 + {C}_{2} N {σ}_{z}^{m} )$$

If the value of ρᵢ cannot be calculated/estimated, it can be approximated by a lookup table for common materials used for road construction. Such lookup table can be generated by testing the material in the laboratory by subjecting the materials to nominal stress conditions.

### Multipass Measurements

The system may include:
a. a distance sensor system located/oriented/configured to (continuously/continually/repeatedly) measure a height of a geomaterial portion (of a construction/mining area) during compaction (of the geomaterial portion);
b. a geolocation unit 118 (“positioning system”) located/mounted/oriented/configured to (continuously/continually/repeatedly) measure/determine a geolocation of the geomaterial portion so that the height of the geomaterial portion at same location can be measured each time to generate a first, second and subsequent measured heights (thus the height at a location is measured after every pass, and height measured at each pass is subtracted from the previous pass to calculate the deformation from each pass); and
c. an electronic processing configured to (continuously/continually/repeatedly):
   i. receive signals from the distance sensor system representing the first and second measured heights of the geomaterial portion after respective first and second compactions, and
   ii. automatically generate a (numerical) estimate/measure representing a geomaterial layer property (e.g., density, stiffness, modulus, energy during compaction, or layer thickness) of the geomaterial portion based on:
      1. the first and second measured heights (including determining/estimating the deformation from a numerical difference between the first and second measured heights); and
      2. a pre-defined relationship/model representing a relationship between (surface) deformation values and geomaterial layer properties.

The system 100 may thus address the problem of inclination, i.e., a non-perpendicular orientation, between the distance sensor beam and the geomaterial portion such that the raw range measurement is too large by about 1/cos(α).)

### Method/Process

The system 100 is configured to perform a method 1100 (or “process”), which includes the estimation of geomaterial layer property values during compaction using sensing and modelling. The estimation of geomaterial density may be referred to as a “proximal” estimation/measurement because the distance sensor system is arranged/operated close/near to the geomaterial surface but not touching the geomaterial surface. The method 1100 includes the data-processing methods and the ML training method.

As shown in FIG. 11, the method 1100 may include:
a. the electronic processing system 106 (which may include a remote/cloud server) receiving deformation measurements from the distance sensor system (e.g., laser sensors) (1102);
b. the electronic processing system 106 receiving geolocation measurements of the respective deformation measurements from a geolocation system mounted on the platform/compactor with the distance sensor system (e.g., a GPS) (1104):
c. the electronic processing system 106 receiving the motion/orientation measurements of the respective deformation measurements (e.g., from the orientation unit 116 and the accelerometer(s) 122) (1106,1108);
d. the electronic processing system 106 storing the received data representing the deformation measurements, the corresponding geolocation measurements and motion/orientation measurements (1110);
e. the electronic processing system 106 pre-processing the received data to filter out the noise in the signal correction method/process (1112);
f. the electronic processing system 106 applying the inputs to the pre-defined relationship/model (1114); and
g. the electronic processing system 106 delivering the estimated geomaterial layer property/density values corresponding to the deformation measurements (and their geolocations) (1116).

As shown in FIG. 9, the signal correction method/process includes the following automatic sub processes:
a. receiving the motion/orientation signals, including:
   i. signals representing vibrations/movement of the movable platform (“vibration signals”), e.g., from the accelerometers 122 (902), and
   ii. signals representing orientations of the movable platform (“orientation signals”), e.g., from the orientation unit 116 (904);
b. receiving the deformation signals (906);
c. the pre-processing to mitigate the electrical noise (908,910,912) as described hereinbefore
d. double integration of the vibration signals (914);
e. high-pass filtering of the deformation signals (916), e.g., at 3 Hz;
f. low-pass filtering of the deformation signals (918), e.g., at 3 Hz;
g. the removal of vibration noise (as described hereinbefore) from the deformation signals based on the (double-integrated de-noised) vibration signals and the (high-pass filtered) deformation signals (920):
h. the inclination correction and/or the roll correction (as described hereinbefore) of the deformation signals based on the (de-noised) orientation signals and the (low-pass filtered) deformation signals (920); and
i. the generating of the estimate/measure of the geomaterial layer property of the geomaterial portion based on: the measured deformation; a (numerical) correction based on the measured motion/orientations; and a pre-defined relationship/model that associates (surface) deformation values with geomaterial layer property values (922).

The method/process 1100 (performed automatically by the system 100, also for estimating/measuring properties, including density, of a geomaterial layer 102 due to compaction by a compactor 104) may include:
a. (continuously/continually/repeatedly) measuring a (surface) deformation Δ (or height change) of a geomaterial portion (of a construction/mining area) due to compaction of the geomaterial portion (including measuring during compaction of the construction/mining area):
b. (continuously/continually/repeatedly) receiving signals (from the distance sensor system 302) representing the measured deformation (“deformation signals”); and
c. (continuously/continually/repeatedly) automatically generating a (numerical) estimate/measure of a geomaterial layer property (e.g., density, stiffness, modulus during compaction, or layer thickness) of the geomaterial portion based on:
   1. the measured deformation (including continuously/continually/repeatedly during the compaction of the construction/mining area); and
   2. a pre-defined relationship/model that associates (surface) deformation values with geomaterial layer property values (e.g., density values, stiffness values, modulus values during compaction, or layer thicknesses), e.g., including a correlation relationship/model and/or a trained machine-learning model.

The measuring of the deformation may be at a distance from/proximate to the geomaterial portion.

The method 1100 may include (continuously/continually/repeatedly) measuring motions/orientations of a movable platform synchronously with the measuring of the deformation.

The method 1100 may include (continuously/continually/repeatedly):
a. receiving signals (from the motion/orientation sensor system) representing the measured motions/orientations (“motion/orientation signals”), wherein the motion/orientation signals are synchronous with the deformation signals, and
b. automatically generating the estimate/measure of the geomaterial layer property of the geomaterial portion based on:
   i. the measured deformation;
   ii. a (numerical) correction based on the measured motion/orientations; and
   iii. a pre-defined relationship/model that associates (surface) deformation values with geomaterial layer property values (e.g., density, stiffness, modulus during compaction, or layer thickness); and

The method 1100 may include (continuously/continually/repeatedly) measuring/determining a geolocation (representing latitude and longitude in coordinates of the construction/mining area) of the geomaterial portion synchronously with the measuring of the deformation.

The method 1100 may include (continuously/continually/repeatedly):
a. measuring/determining the geolocation of the geomaterial portion so that the height of the geomaterial portion at same location can be measured after respective first and second compactions to generate at least a first measured height and a second measured height (thus the height at a location is measured after every pass, and height measured at each pass is subtracted from the previous pass to calculate the deformation from each pass);
b. receive signals (from the distance sensor system 302) representing the first and second measured heights of the geomaterial portion after respective first and second compactions, and
c. automatically generate a (numerical) estimate/measure of the geomaterial layer property (e.g., density, stiffness, modulus during compaction, or layer thickness) of the geomaterial portion based on:
   i. the first and second measured heights (including determining/estimating the deformation from a numerical difference between the first and second measured heights); and
   ii. the pre-defined relationship/model representing a relationship between (surface) deformation values and geomaterial layer properties.

The method 1100 may include (continuously/continually/repeatedly):
a. generating a coded/color-coded map 500 of geomaterial layer property/density values of a plurality of geomaterial portions (and thus up to the entire construction/mining area) based on the geolocations and their respective geomaterial layer property values; and
b. displaying the coded/color-coded map 500 (to an operator of the compactor) to show when the compaction has reached a specified level (for the construction/mining area).

The measuring of the deformation method may include (continuously/continually/repeatedly):
a. automatically measuring a first height of the geomaterial portion before compaction (of the geomaterial portion);
b. automatically measuring a second height of the geomaterial portion after the compaction (of the geomaterial portion); and
c. determining the measured deformation from a difference between the measured first height and the measured second height.

The method 1100 may include generating signals representing vibrations/movement of the movable platform (e.g., roller), including the rigid body movement of the movable platform.

The method 1100 may include generating signals representing orientations of the movable platform (e.g., roller), including the rigid body movement of the movable platform.

### Experimental Examples

In an experimental example, a large-scale soil box (dimensions: 4 m*7 m*0.8 m) provided the construction/mining area.

In an experimental example, using the 1D relationship, the estimated density from the system and method described herein matched with ‘ground truth’ density measured by NDG with an R²=0.8.

The tests involve placement and compaction of granular soil in layers with compacted thickness ranging from 100 mm to 150 mm. Two test sites were designated for the testing and the data collection, Test site 1. The material compacted throughout this study was characterised as sand with silty fines. The geotechnical properties included: Specific gravity (GS) of 2.70, Median diameter (D₅₀) of 0.32 mm, MDD standard of 1.96 Mg/m³, OMC standard of 9.8(%), the Optimum degree of saturation of 70%, Percentage passing the No. 200 sieve of 21, and a USCS classification of SM. The compaction characteristics of the material were studied with three different Proctor energies (standard=E_{proc}, modified=4.5 E_{proc} and reduced=0.6 E_{proc}, where, E_{proc}=594.5 KJ/m³]) as shown in FIG. 12.

Site 1 was an indoor facility. The site included fabricating a wooden box with dimensions (7.5 m in length, 4 m in width and 0.8 m in height) and an additional open area for the ramp for movement of the roller into the box. Site 2 was an outdoor test condition and almost same size as site 1, with test area dimension (0.5 m (depth)×5 m (width)×8 m (length)). The experimental plan included compacting layers of soil by the instrumented roller for data collection. For site 1, five layers of 100 mm were tested, and the material was compacted using a 1.5 t roller (e.g., Roller 1 with details set out in Appendix B). For site 2, three layers with thickness increased to 150 mm were tested, as a heavier roller was used (e.g., Roller 2 with details set out in Appendix B). Both the rollers were double drum vibratory rollers, where both the drums can be vibrated simultaneously and separately. The test procedure included: (a) placing the material using a bobcat: (b) spreading the material manually using shovels and rakes and levelling using a bubble level: (c) compacting the material using the instrumented roller; and (d) in-situ tests for material density and modulus measurement. The test procedure included the following steps:
[Step 1] The material was conditioned at an appropriate moisture content (8% w/w) for site 1 and covered with a tarp for storage.
[Step 2] The material was placed into the test setup using a bobcat and was spread as evenly as possible.
[Step 3] Using shovels and rakes, the material was spread, and then levelled using a large spirit level to get a smooth finish.
[Step 4] Before compaction, measurements were taken, including density and moisture measurement using a sand cone apparatus, NDG and level measurement at 15 points using staff and level to get the surface elevation of the material. The measurement of modulus was taken using light weight deflectometer (LWD).
[Step 5] The instrumentation system was checked, the signal was zeroed, and the DAQ was kept running.
[Step 6] The instrumented roller was then used to compact the material: the whole width was divided into three lanes (A, B and C).
[Step 7] The front roller was set to vibration in the forward pass, whereas the vibration was switched off while reversing to study the impact of vibration.
[Step 8] After every two passes (one forward and one reverse), all the measurements mentioned in Step 4 were repeated.
[Step 9] The data from the instruments of each lane and pass were saved to the computer in separate files.
[Step 10] The density data from sampling (sand cone test) were adopted to decide the end of compaction. At the end of compaction, Nuclear Density Gauge (NDG) was also carried out for comparison with sand cone apparatus and LWD tests were carried to measure the layer modulus.
[Step 11] After compacting one layer, the material for the subsequent layer was placed, and the Steps 3 to 10 above were repeated.

As shown in FIG. 13, the majority of the deformation occurred in the first couple of passes, and then the value plateaus out. Validation of the measurement of the deformation using the laser systems was done by comparing it with deformation measured by level and staff, e.g., as shown in FIG. 14, which is only plotted for Lane C. As shown in FIG. 11, the deformation measured through the laser agreed reasonably well with the deformation measured using level and staff for all the points on Lane A and C. As shown in FIG. 15, the correlation between measured density using NDG and density estimated using the laser deformation measurements was very high, with R²=0.8.

### Implementations

In one or more implementations, the system and method described herein may:
a. estimate the geomaterial layer properties (e.g., density) proximally/non-destructively, in real time, and without sampling:
b. provide quick and real-time estimations of the geomaterial layer properties during compaction:
c. cover the entire area to be assessed in contrast to discreet measurements:
d. use either a vibratory or a static roller:
e. alert the user about the problematic or under-compacted areas/portions on the basis of the geomaterial layer property/density, e.g., when used for QA/QC purposes, alerting the user about problematic or under-compacted area based on measured geomaterial layer properties/density:
f. indicate compliance with performance-based specifications, end-result specifications, method specifications: the end-result specifications are relevant when the compaction takes place until the material has achieved the required density or value of another geomaterial layer property: if measurements of geomaterial layer properties are not possible because of very large size materials being present, the method specification is relevant, which requires compaction until a selected compaction (deformation) threshold is reached—and the system and method described herein may improve on previous methods that relied on contractors visually checking whether the deformation had reached the threshold and/or
g. indicate compliance during ‘proof rolling’, which is carried out in some parts of the world after the end of the compaction to check if the material has been compacted sufficiently: proof rolling involves loading the material manually using a water truck or a suitable vehicle and checking the deformation visually—and the system and method described herein may improve on previous methods that relied on contractors visually checking whether the deformation had reached the threshold.

### Interpretation

The presence of “/” in a FIG. or text herein is understood to mean “and/or”, i.e., “X/Y” is to mean “X” or “Y” or “both X and Y”, unless otherwise indicated.

The FIGs. included herewith show aspects of non-limiting representative embodiments in accordance with the present disclosure, and particular structural elements shown in the FIGs. may not be shown to scale or precisely to scale relative to each other. The depiction of a given element or consideration or use of a particular element number in a particular FIG. or a reference thereto in corresponding descriptive material can encompass the same, an equivalent, an analogous, categorically analogous, or similar element or element number identified in another FIG. or descriptive material associated therewith. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “substantially” can indicate a percentage greater than or equal to 90%, for instance, 92.5%, 95%, 97.5%, 99%, or 100%.

Many modifications will be apparent to those skilled in the art without departing from the scope of the present invention.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

APPENDIX A
EXAMPLE INSTRUMENT DETAILS

| Instrument | Laser | IMU | Accelerometer |
|---|---|---|---|
| Manufacturer | OMRON | PEPPERL + FUCHS | Brũel & Kjær |
| | Corporation | | |
| Model | OM70- | IMU360D-F99-B20- | 4506-B-003 |
| | L1000.HV0700.VI | V15 | |
| Sampling Frequency | 2.5 kHz | 800 Hz (100 Hz per | 2 kHz |
| (Maximum) | | element) | |
| Rated capacity/ | 100-1000 mm | acceleration: ±2 g | ±14 g |
| Measuring range | | inclination: 0-360° | |
| | | rotation rate: ±250°/s | |
| Sensitivity/Resolution | 3-63 μm | acceleration: 0.001 g | 490 ± 10% mV/g |
| | | inclination: 0.01° | |
| | | rotation rate: 0.01°/s | |
| Output circuit | Analog and RS 485 | CAN bus with SAE | Analog |
| | | J1939 protocol | |
| Voltage supply range | +15 to +28 | 0 to +30 | +24 to +30 |
| (VDC) | | | |
| Output signal | +4 to +20 mA/0 to | Not Applicable | −10 to +10 |
| (VDC) | +10 | | |
| Temperature Error | 0.065% So/K | max. ±1.5° at +15 | X: 0.15, Y, Z: 0.12 |
| | (−10 to 50° C.) | to +85° C. | %/° C. (−54 to |
| | | | +100° C. |
| Dimensions, mm | 26 × 55 × 74 | 37.5 × 45 × 65 | 17 × 17 × 17 |

APPENDIX B
EXAMPLE ROLLER DETAILS

| | Roller 1 | Roller 2 |
|---|---|---|
| Manufacturer | HAMM | DYNAPAC |
| Model | HD 10C VV | CC1300 |
| Type | Articulated | Articulated |
| | tandem | tandem |
| | roller | roller |
| | with two | with two |
| | vibratory | vibratory |
| | drums | drums |
| Total length (mm) | 2260 | 2725 |
| Width (mm) | 1056 | 1450 |
| Roller drum width (mm) | 1000 | 1300 |
| Drum diameter (mm) | 620 | 802 |
| Speed range (km/h) | 0 to 11 | 0 to 10 |
| Theoretical gradeability | 30% (vibration ON), | 37% |
| | 40% (vibration OFF) | |
| Static Mass (kg) | 1670 | 3900 |
| Static Drum Linear Load, | 8.1/8.6 | 14.5/15.4 |
| front/rear (kg/cm) | | |
| Vibration frequency (Hz) | 52 | 52 |
| Amplitude (mm) | 0.45 | 0.5 |
| Centrifugal force (kN) | 16 | 33 |

1. A system including:
a distance sensor system located/oriented/configured to measure a deformation of a geomaterial portion due to compaction of the geomaterial portion; and
an electronic processing system configured to:
receive signals from the distance sensor system representing the measured deformation, and
automatically generate an estimate/measure of a geomaterial layer property of the geomaterial portion based on:
the measured deformation; and
a pre-defined relationship/model that associates deformation values with geomaterial layer property values (e.g., density values, stiffness values, modulus values, energy values, or layer thicknesses during compaction).

2. The system of claim 1, wherein the pre-defined relationship/model includes a correlation relationship/model and/or a trained machine-learning model.

3. The system of claim 1, wherein the distance sensor system is configured/mounted/arranged to measure the deformation without touching the geomaterial portion.

4. The system of claim 1, wherein the distance sensor system is located/mounted on/to a movable platform.

5. The system of claim 4, including a motion/orientation sensor system mounted/oriented/configured to measure motions/orientations of the movable platform synchronously with the measurements of the distance sensor system.

6. The system of claim 5, wherein the electronic processing system is configured to:
receive signals from the motion/orientation sensor system representing the measured motions/orientations (“motion/orientation signals”), wherein the motion/orientation signals are synchronous with the deformation signals, and
automatically generate the estimate/measure of the geomaterial layer property of the geomaterial portion based on:
the measured deformation;
a correction based on the measured motion/orientations; and
the pre-defined relationship/model.

7. The system of claim 6, wherein the motion/orientation sensor system includes a motion/vibration sensor system, which includes one or more accelerometers attached/fastened to the movable platform.

8. The system of claim 6, wherein the motion/orientation sensor system includes an orientation/angle sensor system which includes an orientation unit attached/fastened to the movable platform.

9. The system of claim 1, including a geolocation unit located/mounted/oriented/configured to measure/determine a geolocation of the geomaterial portion synchronously with the measurements of the distance sensor system.

10. The system of claim 9, wherein the electronic processing system is configured to:
measure/determine the geolocation of the geomaterial portion so that the height of the geomaterial portion at approximately same location can be measured after respective first and second compactions to generate at least a first measured height and a second measured height; and
receive signals from the distance sensor system representing the first measured height and the second measured height after respective first and second compactions, and
automatically generate the estimate/measure of the geomaterial layer property based on:
the first and second measured heights; and
the pre-defined relationship/model.

11. The system of claim 10, wherein the electronic processing system is configured to generate/display a coded/color-coded map of the estimated/measured geomaterial layer property values of a plurality of geomaterial portions based on the geolocations and their respective geomaterial layer property values, optionally wherein the electronic processing system includes a visible display to display the coded/color-coded map.

12. The system of claim 1, wherein the distance sensor system includes:
at least one first distance sensor located/oriented/configured to measure a first height of the geomaterial portion before compaction of the geomaterial portion; and
at least one second distance sensor located/oriented/configured to measure a second height of the geomaterial portion after the compaction of the geomaterial portion.

13. The system of claim 12, wherein the distance sensor system and/or the electronic processing system is configured to determine the measured deformation from a difference between the measured first height and the measured second height.

14. The system of claim 12, wherein the first distance sensor is configured for attachment to a first side of a compactor element, and wherein the second distance sensor is configured for attachment to a second side of the compactor element, such that the first distance sensor can measure the first height before the compaction and the second distance sensor can measure the second height after the compaction.

15. A method/process including:
a. measuring a deformation of a geomaterial portion due to compaction of the geomaterial portion;
b. receiving signals representing the measured deformation; and
automatically generating an estimate/measure of a geomaterial layer property of the geomaterial portion based on: the measured deformation; and a pre-defined relationship/model that associates deformation values with geomaterial layer property values.
